(19) United States
(12) Patent Application Publication
(10) Pub. No.: US 2023/0100410 A1
(43) Pub. Date: Mar. 30, 2023

(54) ENDOSCOPE, REPAIRING METHOD FOR ENDOSCOPE, MANUFACTURING METHOD FOR ENDOSCOPE, AND MANUFACTURING METHOD FOR INSERTION SECTION OF ENDOSCOPE

(71) Applicant: OLYMPUS CORPORATION, Tokyo (JP)

(72) Inventor: Yoshimi Yamamoto, Tokyo (JP)

(73) Assignee: OLYMPUS CORPORATION, Tokyo (JP)

(21) Appl. No.: 17/961,621

(22) Filed: Oct. 7, 2022

Related U.S. Application Data
(63) Continuation of application No. PCT/JP2021/014524, filed on Apr. 5, 2021.

(30) Foreign Application Priority Data
Apr. 9, 2020 (JP) 2020-070153

(51) Int. Cl. A61B 1/00

(52) U.S. Cl. CPC A61B 1/00071; A61B 1/0011

(57) ABSTRACT

An endoscope includes, in an insertion section, a bending tube covered with a first skin, a flexible tube covered with a second skin, a thread wound around a joining section of the first skin and the second skin, first resin covering the thread and having a modulus of elasticity of 0.5 GPa or more and 5 GPa or less, and second resin covering the first resin, extended from both ends of the first resin respectively to ranges of 100 μm or more, and having a modulus of elasticity of 0.3 MPa or more and 100 MPa or less.

### CROSS REFERENCE TO RELATED APPLICATION

This application is a continuation application of PCT/JP2021/014524 filed on Apr. 5, 2021 and claims benefit of Japanese Application No. 2020-070153 filed in Japan on Apr. 9, 2020, the entire contents of which are incorporated herein by this reference.

### BACKGROUND OF THE INVENTION

### 1. Field of the Invention

The present invention relates to an endoscope including an insertion section in which a fixing member is wound around a joining section of two members, a repairing method for an endoscope including an insertion section in which a fixing member is wound around a joining section of two members, a manufacturing method for an endoscope including an insertion section in which a fixing member is wound around a joining section of two members, and a manufacturing method for an insertion section of an endoscope including the insertion section in which a fixing member is wound around a joining section of two members.

### 2. Description of the Related Art

Japanese Patent No. 5197885 discloses an endoscope in which a thread is wound around, as a fixing member, a joining section of a distal end portion and a bending tube and an adhesive is applied to the thread.

### SUMMARY OF THE INVENTION

An endoscope in an embodiment includes, in an insertion section: a first member covered with a first skin; a second member covered with a second skin; a fixing member wound around a joining section of the first skin and the second skin; first resin covering the fixing member and having a modulus of elasticity of 0.5 GPa or more and 5 GPa or less; and second resin covering the first resin, extended from both ends of the first resin respectively to ranges of 100 μm or more, and having a modulus of elasticity of 0.3 MPa or more and 100 MPa or less.

A repairing method for an endoscope in an embodiment, an insertion section of the endoscope including a first member covered with a first skin, a second member covered with a second skin, a fixing member wound around a joining section of the first skin and the second skin, first resin covering the fixing member, and second resin covering the first resin, extended from both ends of the first resin respectively to ranges of 100 μm or more, and having a modulus of elasticity smaller than a modulus of elasticity of the first resin, the repairing method including: cutting into an interface between the first skin and the second resin while bringing a plane side of an edge of a single-edged knife into contact with the first skin; peeling the second resin from the first skin using the knife; cutting into an interface between the second skin and the second resin while bringing the plane side of the edge of the single-edged knife into contact with the second skin; peeling the second resin from the second skin using the knife; and bending the joining section of the first skin and the second skin to thereby peel the first resin from the first skin.

A manufacturing method for an endoscope includes: covering a first member of an insertion section with a first skin; covering a second member of the insertion section with a second skin; winding a fixing member around a joining section of the first skin and the second skin; covering the fixing member with first resin having a modulus of elasticity of 0.5 GPa or more and 5 GPa or less; and covering the first resin with second resin having a modulus of elasticity of 0.3 MPa or more and 100 MPa or less and extending the second resin from both ends of the first resin respectively to ranges of 100 μm or more.

A manufacturing method for an insertion section of an endoscope includes: covering a first member of the insertion section of the endoscope with a first skin; covering a second member of the insertion section consecutively connected to the first member with a second skin; winding a fixing member around a joining section of the first skin and the second skin; covering the fixing member with first resin having a modulus of elasticity of 0.5 GPa or more and 5 GPa or less; and covering the first resin with second resin having a modulus of elasticity of 0.3 MPa or more and 100 MPa or less and extending the second resin from both ends of the first resin respectively to ranges of 100 μm or more.

### BRIEF DESCRIPTION OF THE DRAWINGS

FIG. 1 is a diagram showing a configuration of an endoscope in an embodiment;

FIG. 2 is an exterior view of a joining section of the endoscope in the embodiment;

FIG. 3 is a partial sectional view taken along a III-III line in FIG. 2;

FIG. 4 is a partial sectional view taken along a IV-IV line in FIG. 2; and

FIG. 5 is a sectional view for explaining a method of disassembling the endoscope in the embodiment.

### DETAILED DESCRIPTION OF THE PREFERRED EMBODIMENTS

### First Embodiment

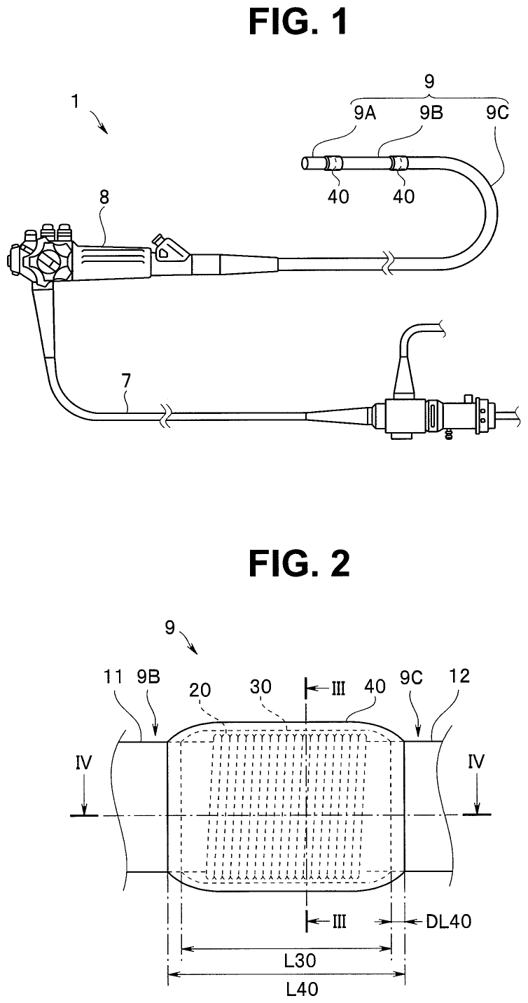

As shown in FIG. 1, an endoscope 1 in a first embodiment includes an insertion section 9, a cross section of which orthogonal to a longitudinal direction is circular, an operation section 8 disposed at a proximal end of the insertion section 9, and a universal cord 7 extending from the operation section 8. The insertion section 9 includes a distal end portion 9A, a bending tube 9B, which is a first member, joined to a proximal end of the distal end portion 9A, and a flexible tube 9C, which is a second member, joined to a proximal end of the bending tube 9B. A use of the endoscope 1 may be either a medical use or an industrial use. In the insertion section 9, the bending tube 9B, which is the first member, and the flexible tube 9, which is the second member, are consecutively connected.

Note that, in the following explanation, drawings based on respective embodiments are schematic. Relations between thicknesses and widths of respective portions, ratios of the thicknesses of the respective portions, and the like are different from real ones. Portions having relations and ratios different from one another are included among the drawings. Illustration of and imparting of signs to some of components are omitted.

A distal end rigid portion is disposed at the distal end portion 9A of the insertion section 9. The distal end rigid portion, a cross section of which is circular, is, for example, a substantially cylindrical housing made of stainless steel. For example, an image pickup apparatus is disposed at the distal end rigid portion. An outer circumference of the distal end rigid portion is covered with a distal end skin made of resin.

The bending tube 9B, a cross section of which is circular, includes a plurality of bending pieces, a mesh tube that covers outer circumferences of the plurality of bending pieces, and a first skin 11 covering an outer circumference of the mesh tube. The plurality of bending pieces are turnably connected. The first skin 11 is an outer skin tube that covers the mesh tube. The bending tube 9B bends, for example, in upward, downward, left, and right directions according to operation of the operation section 8.

The elongated flexible tube 9C, a cross section of which is circular, includes a spiral tube, a mesh tube, and a second skin 12. The spiral tube is manufactured by winding a metal belt-like plate in a spiral shape. The spiral tube is covered with the mesh tube. The mesh tube is manufactured by knitting a polymeric material or a metal element wire in a tube shape. The second skin 12 is an outer skin tube that covers the mesh tube.

The first skin 11 and the second skin 12 are made of, for example, fluororubber. The first skin 11 and the second skin 12 may be made of different materials.

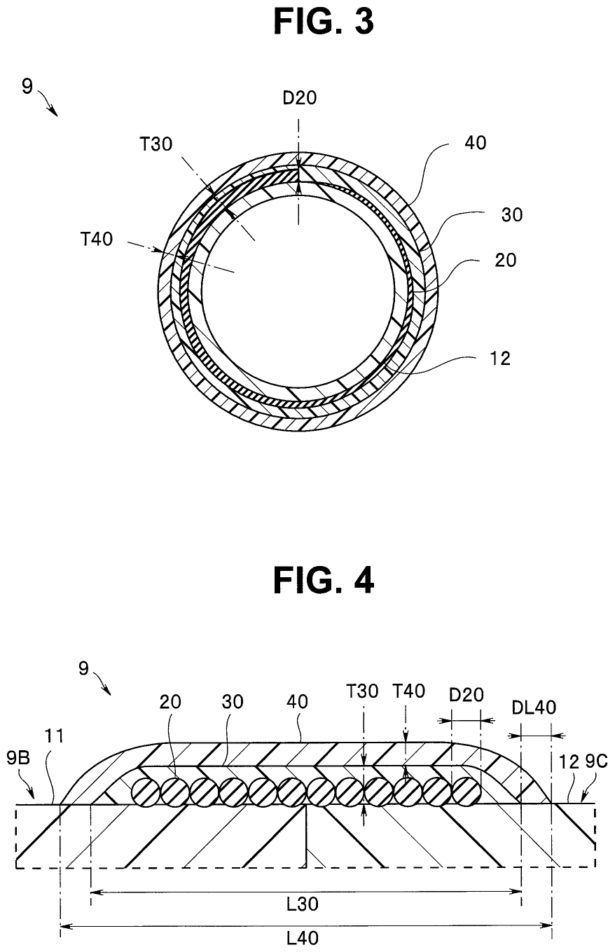

As shown in FIG. 2 to FIG. 4, a thread 20, which is a fixing member, is wound around a joining section of the bending tube 9B and the flexible tube 9C, in other words, a joining section of the first skin 11 and the second skin 12. The thread 20 is covered with first resin 30. The first resin 30 is covered with second resin 40. Note that, in the following explanation, the flexible tube 9C may be the first member and the bending tube 9B may be the second member. The distal end portion 9A and the bending tube 9B are joined by substantially the same method as the joining of the bending tube 9B and the flexible tube 9C. Note that, in FIG. 3, a configuration further on an inner side than the second skin 12 is not shown.

The thread 20 is not contracted and plastically deformed by stream used for sterilization of the endoscope and further has breaking elongation of 10% or less. The thread 20 is a silk thread having a diameter D20 of 200 μm. The diameter D20 of the thread 20 is preferably 10 μm or more and 500 μm or less. If the diameter D20 is equal to or larger than the range, watertightness between the bending tube 9B and the flexible tube 9C can be sufficiently kept. If the dimeter D20 is equal to or smaller than the range, a diameter of the insertion section 9 is not large. Note that the thread 20 may be manufactured from an aramid fiber, a polyarylate fiber, a poly(p-phenylenebenzobisoxazole) fiber, or a carbon fiber or may be manufactured by binding or knitting the fiber explained above.

The thread 20 is covered with the first resin 30. In other words, thickness T30 of the first resin 30 is thickness of 250 μm larger than the outer diameter D20 of the thread 20. The first resin 30 is an adhesive made of epoxy resin, a modulus of elasticity E30 of which is 2 GPa. If the thickness T30 is larger than the outer diameter D20, since the first resin 30 can completely cover the thread 20, the wound thread 20 can be firmly fixed. Note that, for example, when a cross section of the thread 20 is a square, the outer diameter D20 is equivalent to length of a side.

The first resin 30 is covered with the second resin 40. The second resin 40 is made of urethane resin, thickness T40 of which is 250 μm and a modulus of elasticity E40 of which is 50 MPa.

The modulus of elasticity E is a storage elastic modulus measured using a viscoelastic spectrometer (manufactured by Rheometric Scientific F. E. Ltd., RSA-II). Measurement conditions are frequency of 1 Hz, sample thickness of 1 mm, weighting of 100 g, and temperature of 25° C.

As shown in FIG. 2 and FIG. 4, length L30 of the first resin 30 in a longitudinal direction of the insertion section 9 is 10 mm and length L40 of the second resin 40 in the longitudinal direction of the insertion section 9 is 16 mm. In other words, lengths DL40 of both ends of the second resin 40 being in contact with the first skin 11 or the second skin 12 are respectively 3 mm. In other words, the second resin 40 covers the entire surface of the first resin 30 and is extended from both ends of the first resin 30 respectively by 3 mm.

L40=L30+DL40×2

Since the endoscope 1 having the configuration explained above is excellent in shock resistance and bending durability, the endoscope 1 has high reliability and is easy to repair.

As evaluation of the shock resistance, an iron ball having weight of 1 kg was repeatedly dropped on the second resin 40 in the joining section of the endoscope 1 from height of 10 cm and presence or absence of a crack of the second resin 40 was visually checked. When no crack occurred even if the iron ball was dropped one hundred times, the shock resistance was evaluated as “BEST”. When a crack was caused by dropping the iron ball fifty times or more and less than one hundred times, the shock resistance was evaluated as “GOOD”. When a crack was caused by dropping the iron ball less than fifty times, the shock resistance was evaluated as “BAD”.

As evaluation of the bending durability, the endoscope 1 was repeatedly bent at an angle of 90 degrees centering on the joining section of the bending tube 9B and the flexible tube 9C and presence or absence of peeling of an end portion of the second resin 40 was visually checked. When the end portion of the second resin 40 was not peeled even if the endoscope 1 was bent thirty thousand times, the bending durability was evaluated as “BEST”. When the end portion of the second resin 40 was not peeled even if the endoscope 1 was bent ten thousand times or more and less than thirty thousand times, the bending durability was evaluated as “GOOD”. When the end portion of the second resin 40 was peeled by bending the endoscope 1 less than ten thousand times, the bending durability was evaluated as “BAD”.

Both of the shock resistance and the bending durability of the endoscope 1 was “BEST”.

### <Repairing Method for the Endoscope>

When the endoscope 1 is repaired, the first resin 30 and the second resin 40 are peeled and the thread 20 is cut, whereby the bending tube 9B and the flexible tube 9C are separated.

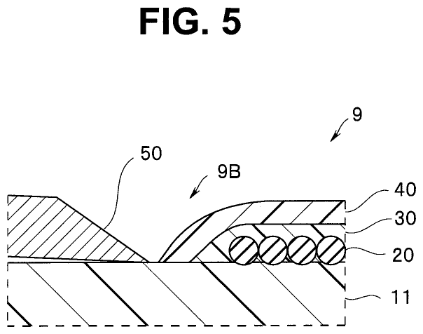

As shown in FIG. 5, a process for cutting into an interface between the first skin 11 and the second resin 40 while bringing a plane side of an edge of a single-edged knife 50 into contact with the first skin 11 is performed. Subsequently, the second resin 40 is peeled from the first skin 11 up to an interface between the first skin 11 and the first resin 30 using the knife 50.

Note that, since a cross section of the joining section is circular, only a part of the second resin 40 can be peeled by performing the work explained above once. The second resin 40 is peeled over an entire circumference of the joining section by repeating peeling work for the second resin 40 after rotating the joining section a little.

The second resin 40 is peeled from the second skin 12 as well by the same method as the peeling method from the first skin 11. The second resin 40 may be peeled from the first skin 11 after being peeled from the second skin 12.

The plane side of the edge of the knife 50 is preferably a concave curved surface and is more preferably a concave surface having substantially the same curvature radius as an outer diameter of the insertion section 9.

After the second resin 40 is peeled from the first skin 11 and the second skin 12, the joining section of the bending tube 9B and the flexible tube 9C is repeatedly bent at an angle of, for example, 90 degrees or more, whereby the first resin 30 is peeled from the first skin 11 and the second skin 12. In other words, since the first resin 30 having the large modulus of elasticity E30 is hardly elastically deformed, the first resin 30 cannot be deformed following deformation of the joining section. Accordingly, a crack occurs in the first resin 30 not having a strong adhesive force and the first resin 30 is peeled from the joining section in pieces.

Note that a pipe sleeve made of a rigid member, for example, a metal tube may be disposed in the joining section for reinforcement. In other words, the thread 20 may be wound around a pipe sleeve that covers a part of the first skin 11 and a part of the second skin 12. In this case, since a skin around the pipe sleeve is greatly elastically deformed by cutting in an end portion of the first resin 30 covered with the second resin 40 using the knife 50 and then bending the end portion of the first resin 30, the first resin 30 that is hardly elastically deformed is peeled from the skin.

The second resin 40 in a region covering the first resin 30 is peeled together with the first resin 30. When the second resin 40 remains on the first skin 11 or the second skin 12, the remaining resin is peeled from the joining section using, for example, the knife 50.

With the repairing method, peeling of the resin is easy and the skin is less easily damaged.

However, when the first skin 11 and the second resin 40 are firmly bonded more than necessary, a large amount of a residue of the second resin 40 is present on a surface of the first skin 11 and the first skin 11 is damaged. When a large amount of the second resin 40 remains on the first skin 11, the second resin 40 needs to be removed and work takes time. When the first skin 11 is damaged, the first skin 11 needs to be replaced.

As evaluation of repairability, after the second resin 40 was peeled, presence or absence of a residue of the second resin 40 and damage to the first skin 11 are visually checked. When a residue of the second resin 40 was absent, the repairability was evaluated as “BEST”. When a residue of the second resin 40 was present but damage requiring replacement was absent in the first skin 11, the repairability was evaluated as “GOOD”. When damage requiring replacement was present in the first skin 11, the repairability was evaluated as “BAD”.

The repairability of the endoscope 1 was “BEST”. If not damaged, the bending tube 9B and the flexible tube 9C separated for repairing are joined and the thread 20 is wound around the bending tube 9B and the flexible tube 9C and, then, the bending tube 9B and the flexible tube 9C are coated with the first resin 30 and the second resin and reused. Accordingly, cost for repairing the endoscope 1 is low.

As explained above, since the endoscope 1 is excellent in the shock resistance and the bending durability, reliability of the endoscope 1 is high. In the repairing method for the endoscope 1, since peeling of the resin is easy and the skin is less easily damaged, workability is high.

### Modifications of the First Embodiment and Comparative Examples

Since endoscopes in modifications of the first embodiment and endoscopes in comparative examples are similar to the endoscope 1 in the first embodiment, components having the same functions are denoted by the same reference numerals and signs and explanation of the components is omitted. In the respective endoscopes, components not clearly described are the same as the components of the endoscope 1. For example, the modulus of elasticity E40 and the like of the second resin 40 of an endoscope in which only the modulus of elasticity E30 of the first resin 30 is clearly described are the same as the modulus of elasticity E40 and the like of the endoscope 1.

### <Single-Layer Resin>

In an endoscope in a comparative example in which the thread 20 is covered with single-layer epoxy resin having a modulus of elasticity of 3 GPa, thickness of 500 μm, and length of 10 mm, the repairability was “BEST” but the shock resistance and the bending durability were “BAD”. In other words, resin having a high modulus of elasticity is easily cracked and easily peeled.

In an endoscope in a comparative example in which the thread 20 is covered with single-layer urethane resin having a modulus of elasticity of 50 MPa, thickness of 500 μm, and length of 10 mm, the shock resistance and the bending durability were “BEST” but the repairability was “BAD”. In other words, it is not easy to clearly peel resin having a low modulus of elasticity at a repairing time.

### <Moduli of Elasticity of Stacked Two Kinds of Resin>

In an endoscope in a comparative example in which the modulus of elasticity E40 of the second resin 40 is equal to or larger than the modulus of elasticity E30 of the first resin 30, all of the shock resistance, the bending durability, and the repairability were “BAD”.

### <Modulus of Elasticity of the First Resin: E30>

In an endoscope in which the modulus of elasticity E30 of the first resin 30 is less than 0.5 GPa, it is likely that adhesion between the first resin 30 and the first skin 11 is high and peeling of the first resin 30 is not easy. When the modulus of elasticity E30 is less than 0.5 GPa, since fixing of the thread 20 is insufficient, it is likely that water tightness of the bending tube 9B and the flexible tube 9C is insufficient. The modulus of elasticity E30 is preferably 1 GPa or more and particularly preferably 1.5 GPa or more. On the other hand, in an endoscope in which the modulus of elasticity E30 is more than 5 GPa, the first resin 30 is fragile and easily cracked. The modulus of elasticity E30 is preferably 0.5 GPa or more and 5 GPa or less.

### <Modulus of Elasticity of the Second Resin: E40>

In an endoscope in which the modulus of elasticity E40 of the second resin 40 is 100 MPa or less, all of the shock resistance, the bending durability, and the repairability were “GOOD”. Further, in an endoscope in which the modulus of elasticity E40 is 30 MPa or less, all of the shock resistance, the bending durability, and the repairability were “BEST”.

Note that, when the modulus of elasticity E40 of the second resin 40 is less than 0.3 MPa, damage occurs in a skin at a repairing time. Accordingly, the modulus of elasticity E40 is preferably 0.3 MPa or more, 100 MPa or less, and 1 MPa or more and particularly preferably 2 MPa or more.

The first resin 30 is preferably, for example, epoxy resin. The second resin 40 is preferably, for example, acrylic resin, urethane resin, silicone resin, or alkyd resin. In order to obtain the modulus of elasticity explained above, the first resin 30 is preferably thermosetting resin and the second resin 40 is preferably, for example, normal-temperature hardening resin of a two-liquid mixing type. Note that it goes without saying that even resin sufficiently hardening at normal temperature may be subjected to heat treatment to accelerate reaction. However, a heat treatment temperature of the second resin 40 is lower than a hardening treatment temperature of the first resin 30.

### <Thicknesses T30 and T40>

As explained above, since the first resin 30 completely covers the thread 20, the thickness T30 of the first resin 30 is larger than the outer diameter D20 of the thread 20. In other words, the thickness T30 is a distance from the first skin 11 or the second skin 12 to an upper surface of the first resin 30. On the other hand, in an endoscope in which the thickness T40 of the second resin 40 is smaller than 20 μm, since a shock is not absorbed by the second resin 40, it is likely that a crack occurs in the first resin 30. The thickness T40 of the second resin 40 is preferably 20 μm or more.

In an endoscope in a modification in which the thickness T40 of the second resin 40 is 50 μm or more, the shock resistance was “GOOD”. In an endoscope in which the thickness T40 is 150 μm or more, the shock resistance was “BEST”.

However, in an endoscope in which the thickness T40 is larger than 500 μm, a diameter of the insertion section 9 is partially large. Accordingly, the thickness T40 is preferably 500 μm or less and particularly preferably 350 μm or less.

<Lengths L30 and DL40>

Length of a winding section of the thread 20 (a thread winding section) is preferably 2 mm or more and particularly preferably 5 mm or more in order to water-tightly seal the joining section of the bending tube 9B and the flexible tube 9C. The length L30 of the first resin 30 covering the thread 20 is larger than the length of the thread winding section. Note that, when workability of manufacturing and repairing is considered, the length L30 is preferably 20 mm or less and particularly preferably 15 mm or less.

As explained above, the length L40 of the second resin 40 is an added value of the length L30 of the first resin 30 and the length DL40 of both the ends of the second resin 40 being in contact with the first skin 11 or the second skin 12.

In an endoscope in a comparative example in which the length DL40 is smaller than 100 μm, the bending durability was “BAD”. In other words, since adhesion strength of the hard first resin 30 to the soft first skin 11 is insufficient, an end portion of the hard first resin 30 is easily peeled when the length DL40 is small.

An endoscope in which the length DL40 is 100 μm or more, the bending durability was “GOOD”. In other words, this is because an end portion that is a start point of peeling of the first resin 30 is sufficiently covered by the second resin 40 that is soft and has high bonding strength to the first skin 11. Note that the length DL40 is preferably 500 μm or more and particularly preferably 1 mm or more.

On the other hand, in an endoscope in which the length DL40 is too large, it is likely that a large amount of a residue of the second resin 40 occurs at the time of repairing. Accordingly, the length DL40 is preferably 20 mm or less and particularly preferably 10 mm or less.

Note that, when length of the first resin 30 being in contact with the first skin 11 and length of the second resin 40 being in contact with the second skin 12 are different, it goes without saying that both the lengths are within the range explained above.

### <Viscosity>

Viscosity η40 of the second resin 40 when being applied is preferably 0.01 Pa·s or more and 300 Pa·s or less and particularly preferably 0.1 Pa·s or more and 200 Pa·s or less. If the viscosity 140 is the range or more, the second resin 40 does not excessively spread to a periphery of the first resin 30 or thickness of the second resin 40 is not excessively thin. If the viscosity 140 is the range or less, the second resin 40 can be applied to a desired range and the thickness of the second resin 40 is not excessively thick. The applied second resin 40 is changed to a solid by evaporation of a solvent or hardening treatment.

As explained above, it goes without saying that a joining section of the distal end portion 9A and the bending tube 9B has the same configuration as the joining section of the bending tube 9B and the flexible tube 9C explained above. In other words, the distal end portion 9A may be the first member or the second member and the bending tube 9B may be the second member or the first member.

The thread 20 may be covered by three or more layers of resin. For example, the endoscope 1 may further include third resin extended from both the ends of the first resin 30, covered with the second resin 40, and having a modulus of elasticity smaller than the modulus of elasticity of the first resin 30 and larger than the modulus of elasticity of the second resin 40.

### Second Embodiment

It goes without saying that the present invention can be used not only for the repairing method for an endoscope but also for a manufacturing method for an endoscope and a manufacturing method for an insertion section of an endoscope.

A manufacturing method for an endoscope in a second embodiment includes covering a first member of an insertion section of the endoscope with a first skin, covering a second member of the insertion section with a second skin, winding a fixing member around a joining section of the first skin and the second skin, covering the fixing member with first resin having a modulus of elasticity of 0.5 GPa or more and 5 GPa or less, covering the first resin with second resin having a modulus of elasticity of 0.3 MPa or more and 100 MPa or less, and extending the second resin from both ends of the first resin respectively to ranges of 100 μm or more.

A manufacturing method for an insertion section of an endoscope in a modification of the second embodiment includes covering a first member with a first skin, covering a second member consecutively connected to the first member with a second skin, winding a fixing member around a joining section of the first skin and the second skin, covering the fixing member with first resin having a modulus of elasticity of 0.5 GPa or more and 5 GPa or less, covering the first resin with second resin having a modulus of elasticity of 0.3 MPa or more and 100 MPa or less, and extending the second resin from both ends of the first resin respectively to ranges of 100 μm or more.

Note that the fixing member may be a thread, a cross section of which is elliptical, square, or polygonal or may be a belt, a cross section of which is substantially rectangular, or a ring having elasticity.

The present invention is not limited to the embodiments and the like explained above. Various changes, alterations, and the like are possible within a range in which the gist of the present invention is not changed.

The present application is filed based on priority of Japanese Patent Application No. 2020-070153 filed in Japan on Apr. 9, 2020, disclosed contents of which are incorporated in the present specification, the claims, and the drawings.

What is claimed is:

1. An endoscope comprising, in an insertion section:
a first member covered with a first skin;
a second member covered with a second skin;
a fixing member wound around a joining section of the first skin and the second skin;
first resin covering the fixing member and having a modulus of elasticity of 0.5 GPa or more and 5 GPa or less; and
second resin covering the first resin, extended from both ends of the first resin respectively to ranges of 100 μm or more, and having a modulus of elasticity of 0.3 MPa or more and 100 MPa or less.

2. The endoscope according to claim 1, wherein thickness of the second resin is 20 μm or more and 500 μm or less.

3. The endoscope according to claim 1, wherein
the first resin is epoxy resin, and
the second resin is acrylic resin, urethane resin, silicone resin, or alkyd resin.

4. The endoscope according to claim 1, wherein
the first resin is thermosetting resin, and
the second resin is normal-temperature hardening resin.

5. The endoscope according to claim 1, further comprising third resin covering the first resin, extended from both the ends of the first resin, covered with the second resin, and having a modulus of elasticity smaller than the modulus of elasticity of the first resin and larger than the modulus of elasticity of the second resin.

6. The endoscope according to claim 1, wherein
the insertion section includes a distal end portion, a bending tube joined to a proximal end of the distal end portion, and a flexible tube joined to a proximal end of the bending tube,
the first member is the bending tube, and
the second member is the distal end portion or the flexible tube.

7. A repairing method for an endoscope, an insertion section of the endoscope including a first member covered with a first skin, a second member covered with a second skin, a fixing member wound around a joining section of the first skin and the second skin, first resin covering the fixing member, and second resin covering the first resin, extended from both ends of the first resin respectively to ranges of 100 μm or more, and having a modulus of elasticity smaller than a modulus of elasticity of the first resin,
the repairing method comprising:
cutting into an interface between the first skin and the second resin while bringing a plane side of an edge of a single-edged knife into contact with the first skin;
peeling the second resin from the first skin using the knife;
cutting into an interface between the second skin and the second resin while bringing the plane side of the edge of the single-edged knife into contact with the second skin;
peeling the second resin from the second skin using the knife; and
bending the joining section of the first skin and the second skin to thereby peel the first resin from the first skin.

8. A manufacturing method for an endoscope comprising:
covering a first member of an insertion section of the endoscope with a first skin;
covering a second member of the insertion section with a second skin;
winding a fixing member around a joining section of the first skin and the second skin;
covering the fixing member with first resin having a modulus of elasticity of 0.5 GPa or more and 5 GPa or less; and
covering the first resin with second resin having a modulus of elasticity of 0.3 MPa or more and 100 MPa or less and extending the second resin from both ends of the first resin respectively to ranges of 100 μm or more.

9. A manufacturing method for an insertion section of an endoscope comprising:
covering a first member of the insertion section of the endoscope with a first skin;
covering a second member of the insertion section consecutively connected to the first member with a second skin;
winding a fixing member around a joining section of the first skin and the second skin;
covering the fixing member with first resin having a modulus of elasticity of 0.5 GPa or more and 5 GPa or less; and
covering the first resin with second resin having a modulus of elasticity of 0.3 MPa or more and 100 MPa or less and extending the second resin from both ends of the first resin respectively to ranges of 100 μm or more.
